(19) United States
(12) Patent Application Publication
(10) Pub. No.: US 2006/0286549 A1
(43) Pub. Date: Dec. 21, 2006

(54) Microfluidic system for identifying or sizing individual particles passing through a channel

(75) Inventors: Lydia L. Sohn, Oakland, CA (US); Andrea Carbonaro, Berkeley, CA (US); Lucy A. Godley, Chicago, IL (US)

Correspondence Address: GORDON & REES LLP, 101 WEST BROADWAY, SUITE 1600, SAN DIEGO, CA, 92101, US

(73) Assignee: The Regents of the University of California, Oakland, CA (US)

(21) Appl. No.: 11/418,860

(22) Filed: May 5, 2006

Related U.S. Application Data
(60) Provisional application No. 60/678,254, filed on May 6, 2005.

(51) Int. Cl. C12Q 1/70; C12Q 1/68; C12M 1/34

(52) U.S. Cl. 435/5; 435/6; 435/287.1; 977/924

(57) ABSTRACT

An apparatus for characterizing and identifying individual particles, including: an input reservoir; at least one output reservoir; a channel connecting the input reservoir to the at least one output reservoir, wherein the channel is functionalized with at least one molecule selected to interact with a marker on a surface of a particle; a system to move fluid containing the particle from the input reservoir through the channel and into the at least one output reservoir; and a system to measure the period of time during which the particle moves through the channel. The particle may optionally be a cell, the at least one molecule may be a protein functionalized onto the channel to interact with the protein on the surface of the cell so as to slow passage of the target cell through the channel. By measuring the period of time during which the particle takes to move through the channel, the particle can be characterized and thereby identified.

### RELATED APPLICATION

The present application claims priority to U.S. Provisional Patent Application Ser. No. 60/678,254, filed May 6, 2005, herein expressly incorporated by reference in its entirety for all purposes.

### TECHNICAL FIELD

The present invention relates to microfluidic systems and to systems that characterize and/or identify individual particles based upon expression of markers on the surface of the individual particles.

### BACKGROUND OF THE INVENTION

Cell identification in a heterogeneous population is an essential component of basic scientific research to identify particular cell populations as well as a critical element in clinical diagnosis and therapeutic monitoring. Both flow-activated cell sorting (FACS) and magnetic-bead labeling with column separation are used routinely in the characterization of samples for immunologic and/or hematologic applications.

Although both techniques are widely adopted, neither is well suited for performing point-of-care analysis, since both require: labeling and careful manipulation of cells; counting of thousands of cells in volumes on the order of milliliters; and instruments requiring specialized training.

Conventional flow cytometry has made innumerable contributions to clinical medicine and biomedical science, especially in the fields of immunology, cancer biology, and stem cell biology. For example, flow cytometry is used routinely in the clinical diagnosis of the hematologic malignancies; in tumor immunology to define lymphocyte subsets; and in basic research to facilitate cell separations based on the expression of particular proteins or phospholipids at the cell-surface. Apoptosis consists of a complex series of cellular events leading to cell death, but it can be assessed simply by a standard flow cytometric assay. However, because defects in this process are fundamental to the acquisition of chemotherapy resistance of cancer cells, it would be extremely useful to have a reliable and easy method for the detection of apoptotic cells that could be performed at the point of care.

Unfortunately, conventional flow cytometry has its limitations. For example, it requires large, bulky equipment which is technically difficult to operate and requires specialized training. As such, it is not a “portable” technology. In addition, conventional flow cytometry is performed on sample sizes typically on the order of thousands of cells. Therefore, it would be desirable to develop a system to perform flow cytometry in a hand-held device using fewer cells. Such a system could be used to improve upon physicians' ability to detect minimal residual disease states and upon scientists' ability to study cell populations that occur in very small numbers (e.g. stem cells).

### SUMMARY OF THE INVENTION

The present invention includes an apparatus for identifying individual particles, having: an input reservoir; at least one output reservoir; a channel connecting the input reservoir to the at least one output reservoir, wherein the channel is functionalized with at least one molecule selected to interact with a marker on a surface of a particle; a system to move fluid containing the particle from the input reservoir through the channel and into the at least one output reservoir; and a system to measure the period of time during which the particle moves through the channel.

In preferred aspects, the system to measure the period of time during which the particle moves through the channel comprises a system to measure the current and/or voltage changes that result when the particle moves through the channel.

The molecules on the surface of the functionalized channel will interact with the markers on the surface of the particle, thereby slowing the passage of the particle though the channel. The present system thus identifies the particle by determining the period of time it takes for the particle to pass through the channel. As well, the particle could be identified by its size and electrical characteristic, both of which could be reflected in the current and/or voltage across the channel as the particle passes therethrough.

In various aspects of the invention, the particle may include a cell, cell fragment, a colloid, a bacterium, a virus, a fungus, a micelle, a liposome, DNA, RNA, or any oligonucleotide chain. (It is to be understood that the forgoing list is exemplary and is not exhaustive). The molecule that is functionalized into the channel may optionally include a protein, a phospholipid, a sugar, a carbohydrate, a peptidoglycan, DNA, RNA or any oligonucleotide chain. (It is to be understood that the forgoing list is exemplary and is not exhaustive). The marker on the surface of the particle may include a protein, a phospholipid, a sugar, a carbohydrate, a peptidoglycan, DNA, RNA or any oligonucleotide chain. (It is to be understood that the forgoing list is exemplary and is not exhaustive). In one exemplary embodiment, the particle is a cell, and the at least one molecule functionalized onto the surface of the channel is a protein. In one exemplary use of the invention, cancer cells can be individually screened (and sorted for further analysis) on the basis of their cell-surface protein expression.

The system to measure the period of time during which the particle takes to move through the channel may be a system for measuring a change in electrical resistance or current across the channel, such as a Coulter counter.

Optionally, the channel has a width of less than 50 μm, and length of less than 2 cm. (It is to be understood that the forgoing dimensions are merely exemplary, and that the present invention is not limited by such dimensions). However, being a nano or micro scale device, the present system can be fabricated into a unitary block of material such as PDMS, glass, quartz, a plastic substrate, silicon, or a semi-conductor wafer.

In optional embodiments, a particle sorter is also included to sort identified particles into either first or second output reservoirs. It is to be understood that the present invention is not so limited as systems in which the particle may be sorted among three, four or more output reservoirs are also contemplated within the scope of the present invention.

In other optional embodiments, the present device operates in parallel, with a computer controlling the operation of a plurality of the devices. Optional particle sorters may also be included to direct identified particles into different output reservoirs. In addition, the present devices can be operated in series, such that the output of one device may become the input for the next device. As will be shown, “trees” of the present devices can be built.

The present invention also includes an apparatus for determining the size of an individual particle, having: an input reservoir; at least one output reservoir; a channel connecting the input reservoir to the at least one output reservoir, wherein the channel is filled with a conducting fluid; a system to move fluid containing a particle from the input reservoir through the channel and into the at least one output reservoir; a system for measuring a change in electrical resistance across the channel; and a system for correlating the amplitude of the change in electrical resistance across the channel to the size of the particle.

Optional particle sorters can also be included together with this apparatus for determining the size of individual particles. Moreover, a plurality of apparati for determining the size of an individual particle can be operated in parallel or in series. In addition, the exemplary apparatus for determining the size of individual particles can be operated together (in parallel or in series) with the exemplary apparatus for identifying individual particles as described above. In one exemplary embodiment of the invention, the apparatus for determining the size of individual particles is positioned upstream of the apparatus for identifying individual particles, and particles are subsequently binned according to size. As such, the output of the particle sizing device becomes the input for the particle identification device.

An endless variety of combinations of microfluidic systems can be built using various combinations of the present invention. Therefore, it is to be understood that the above descriptions are merely exemplary and are not limiting.

The present invention has a number of advantages over conventional fluid cytometry systems, including but not limit to, the following.

First, the present invention provides label-free and direct signal detection.

Second, the present invention provides improved sensitivity, and extreme rapidity and reproducibility.

Third, the present invention can be used with samples of very few cells (or other particles of interest).

Fourth, the present invention can be easily operated by a lay person/patient, doctor, nurse or other health professional.

Fifth, there is no need for significant manipulations or incubations of cells prior to use with the present invention.

Sixth, the present invention involves low cost electronic detection (as compared to more expensive conventional chemical or optical systems of detection).

The above advantages are exemplary and are not limiting. Other advantages and features of the invention can be seen herein.

### BRIEF DESCRIPTION OF THE DRAWINGS

FIG. 1 is a top plan view of the present invention.

FIG. 2A is an enlarged view of a first (straight) embodiment of the channel.

FIG. 2B is an enlarged view of a second (serpentine) embodiment of the channel.

FIG. 3 is an illustration of two different cells passing through a functionalized channel. (Note: In operation, the cells pass through the channel at different times).

FIG. 4 is an illustration of current change across the channel as the cells of FIG. 3 pass through the channel.

FIG. 5 is an illustration of cells of two different sizes passing through a non-functionalized channel. (Note: In operation, the cells pass through the channel at different times).

FIG. 6 is an illustration of current change across the channel as the cells of FIG. 5 pass through the channel.

FIG. 7 is an illustration of a plurality of devices of the present invention working in parallel.

FIG. 8 is an illustration of a “tree” formed by a plurality of devices of the present invention working in series.

FIGS. 9 to 11 are data produced by successfully operating the present invention.

### DETAILED DESCRIPTION OF THE DRAWINGS

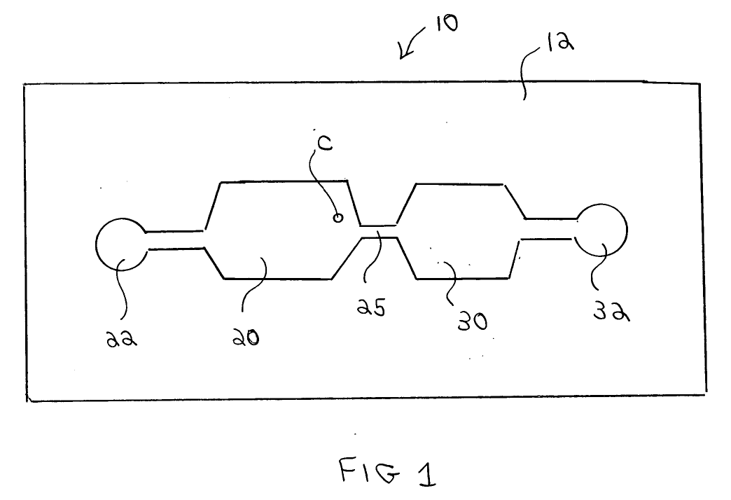

The present invention provides an apparatus for identifying individual particles, as follows. FIG. 1 shows a simplified embodiment of the present invention. Specifically, device 10 comprises an input reservoir 20, an output reservoir 30, and a channel 25 connecting reservoirs 20 and 30.

As can be seen, input reservoir 20, channel 25 and output reservoir 30 may all be fabricated into a unitary block of material 12. Such unitary block of material 12 may be, but is not limited to, PDMS, glass, plastic, quartz, silicon and a semi-conductor wafer.

A system is included to move fluid containing a particle C from input reservoir 20 through channel 25 and into output reservoir 30. In one embodiment, the system to move fluid containing particle C from input reservoir 20 through channel 25 and into output reservoir 30 may be pressure driven. For example, a pressure differential may simply be applied across reservoirs 20 and 30 by increasing the pressure on reservoir 20 as compared to reservoir 30. This may be done by inserting fluid into input 22 or by extracting fluid from output 32.

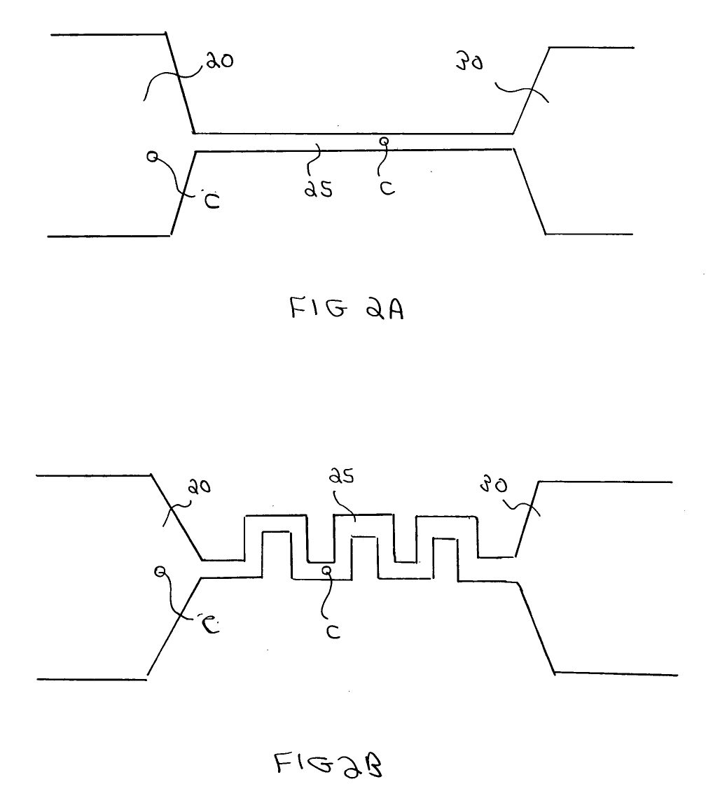

FIGS. 2A and 2B show close-ups of channel 25. In FIG. 2A, channel 25A is straight. In FIG. 2B, channel 25B is serpentine. As will be explained, an advantage of channel 25B is that particles C passing therethrough have an increased likelihood of contacting the walls of channel 25. In various embodiments of the invention, channel 25 has a width of less than 50 μm, and a length of less than 2 cm. In different embodiments, the width of channel 25 may depend upon the sizes of particles P passing therethrough. Some exemplary ranges may be less than 5 μm for platelets, 5-10 μm for red blood cells, 10-15 μm for leukocytes and lymphoblasts; 20 μm for myeloblasts; and 30 μm for monoblasts. Again, it is to be understood that the present invention is not limited to these particular exemplary dimensions.

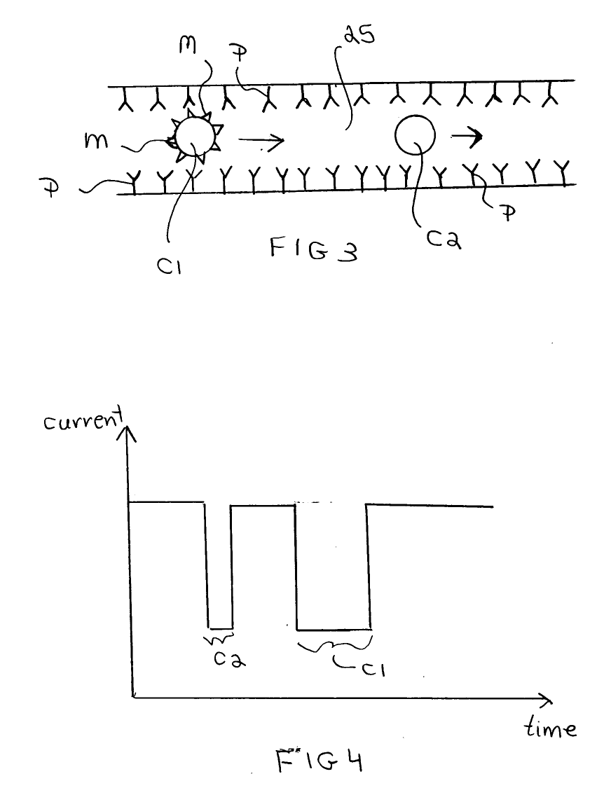

In one aspect of the invention, channel 25 is functionalized with at least one molecule selected to interact with a marker on a surface of a particle passing through channel 25. This is seen in FIG. 3, where two different cells C1 and C2 are moving along through channel 25. (Note: for ease of illustration, the cells are shown together in the channel; however, in accordance with the present invention, the cells pass one-by-one through the channel.) The walls of channel 25 are functionalized with a protein (or other molecule) P. Cell C1 has a particular surface marker M, whereas cell C2 does not have surface marker M.

Fabricating channel 25 from glass, quartz or silicon is particularly advantageous in that these materials well suited to be functionalized with various molecules since it has a high hydrophobic surface (—O—Si(CH₃)₂), which can adsorb proteins by hydrophobic interactions with the non-polar residues of an amino acid chain. Fabricating channel 25 from PDMS is particularly advantageous in that PDMS is flexible and easy to fabricate.

Marker M will interact with protein P such that cell C1 takes a longer period of time to pass through channel 25 (as compared to cell C2 which will pass quickly therethrough). As such, the present invention also includes a system to measure the period of time during which the particle moves through the channel. This system may optionally comprise a system for measuring a change in electrical resistance across the channel. (For example, a micro-Coulter counter that measures current change across channel 25 over time). In this embodiment of the invention, the fluid passing through channel 25 is a conducting fluid.

An exemplary micro-Coulter counter system for electronically detecting the presence of a particle in chamber 25 is described in U.S. Published patent application Ser. No. 10/056,103, to Sohn et. al., entitled “Method And Apparatus For Analysis Of Biological Solutions” incorporated by reference herein in its entirety for all purposes.

Coulter counters typically consist of two fluid-filled reservoirs of particle-laden solution separated by a membrane and connected by a small aperture, pore or channel in that membrane. Particles in the solution are driven through the pore and in doing so, displace conducting fluid and raise the electrical resistance of the pore. By monitoring the changes in electrical current through the pore as individual particles pass from one reservoir to the other, Coulter counters are able to measure the sizes of particles passing through the pore.

In the Sohn system, a device is provided that allows the Coulter principle to be applied to the detection and measurement of particles ranging in size from sub-micron to several microns. The device comprises a conduit through which a liquid suspension of particles to be sensed and characterized can be made to pass, wherein the conduit has an effective electrical impedance which is changed with the passage of each particle therethrough; a liquid handling-system for causing the liquid suspension of particles to pass through the conduit; and a measurement system for sensing the change of electrical impedance in the conduit.

The Sohn system is especially well adapted for use with the present invention. The Sohn system can thus be used to detect the presence of the individual particles in channel 25. By determining the amount of time it takes for each individual particle to pass through channel 25, it is possible to determine that interaction between the functionalized walls of channel 25 and the surface of the particle has occurred. Thus, in accordance with the present invention, it is possible to determine the identity of the particle.

In one embodiment of the invention, the Sohn system is used with a four-terminal measurement being taken on channel 25 to remove both the resistance of the electrodes and the interfacial resistance between the electrodes and the buffer (conducting fluid) solution in channel 25. As a result, the present invention measures solely the resistance of channel 25 and thus is very well suited to measure nanometer-sized changes in colloids due to ligand-receptor binding on the colloid surface.

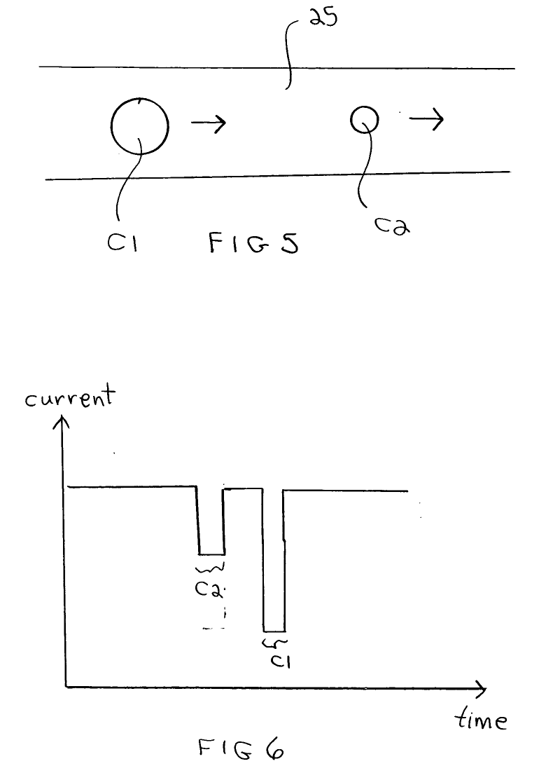

FIG. 4 is an illustration of current change across the channel as the cells of FIG. 3 pass sequentially through the channel. Cells C1 and C2 pass sequentially through channel 25. (Note: for ease of illustration, the cells are shown together in the channel; however, in accordance with the present invention, the cells pass one-by-one through the channel.) The difference in flow rate of the cells C1 and C2 in channel 25 is detected through the difference in current “pulse width” (i.e.: duration of the period of change in electrical resistance across the channel). As can be seen, cells C1 express the marker of interest, and thus have a larger “pulse width” (i.e.: change in electrical resistance across the channel) as compared to that of cells C2. Thus, in accordance with the present invention, the identities of individual cells C1 and C2 can be distinguished from one another by distinguishing between the particular travel times for these cells through channel 25.

It is to be understood that the above description is only exemplary and not limiting. For example, cells C1 and C2 can be replaced by any particle, including, but not limited to cells, cell fragments, colloids, bacteria, viruses, fungi, micelles and liposomes. In addition, although channel 25 may be functionalized with a protein P, it may more generally be functionalized by other molecules, including, but not limited to, a phospholipid, a sugar, a carbohydrate, a peptidoglycan, DNA or RNA or any oligonucleotide chain. Similarly, the marker on the surface of cell C21 may comprise a protein, a phospholipid, a sugar, a carbohydrate, and a peptidoglycan, DNA or RNA or any oligonucleotide chain.

In accordance with the present invention, the particular molecule selected to functionalize the walls of channel 25 is a molecule which interacts with the particular marker on the target particle passing through channel 25. As such, cells C1 that have correspondingly specific cell-surface marker proteins will interact with the coated walls and be retarded in their movement through channel 25, while cells C2 that do not express the marker of interest on the outer surface of the cell membrane will not interact with the functionalized walls and will easily pass through channel 25.

Thus, the present invention can be used to distinguish between cell types Cl from C2 (by identifying cells C1 which take longer to pass through channel 25). In addition, the present invention can be used to identify the presence of a cell C1 in any fluid sample by measuring how long it takes for the cell in channel 25 to move therethrough.

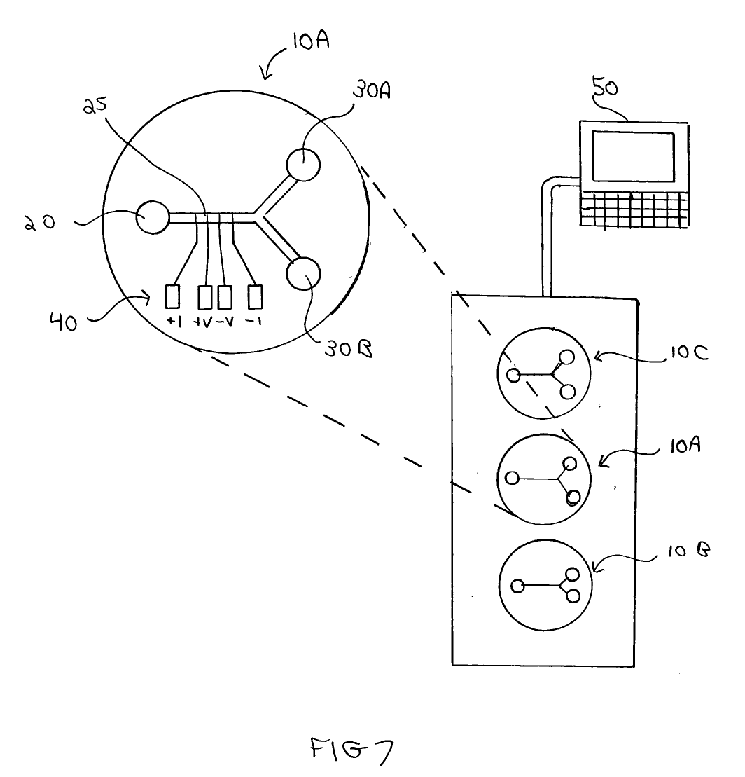

FIGS. 5 and 6 illustrate an alternate embodiment of the invention for determining the size of an individual particle, using the same basic layout as FIGS. 1 and 2A, as follows. System 10 again includes input reservoir 20, channel 25 (filled with a conducting fluid) and output reservoir 30 as described above. Also included are a system to move fluid containing a particle from input reservoir 20 through channel 25 and into output reservoir 30, and a system for measuring a change in electrical resistance across channel 25, as also described above.

However, in this embodiment of the invention, channel 25 is not functionalized as described above (and as shown in FIG. 3). Instead, a system for correlating the amplitude of the change in electrical resistance across channel 20 to the size of the particle is provided, as follows.

As seen in FIG. 5, cells of two different sizes (C1 and C2) are passing along through a non-functionalized channel 25. The larger cell C1 will displace more conducting fluid than the smaller cell C2 when passing therethrough. As seen in FIG. 6, the difference in sizes of cell C1 and cell C2 in channel 25 will be detected through the difference in pulse amplitude. Specifically, larger cells C1 will have a larger “pulse amplitude” (i.e.: amplitude of change in electrical resistance across the channel) as compared to that of smaller cells C2.

Thus, in accordance with the present invention, the sizes of individual cells Cl and C2 can be distinguished from one another by distinguishing between the pulse amplitudes generated by each of cells C1 and C2 passing through channel 25. An advantage of this aspect of the invention is that it operates independently of the travel time of the cell (or any other particle) passing along through channel 25.

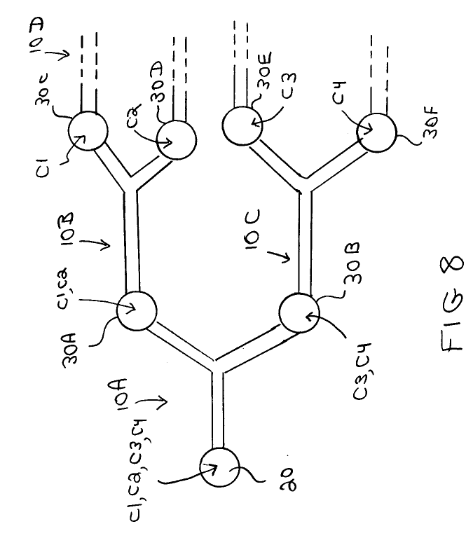

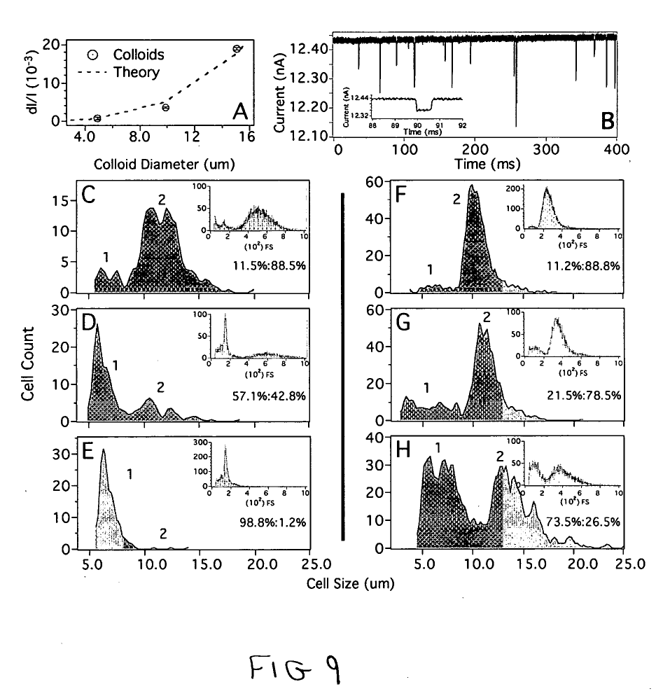

As shown by FIGS. 7 and 8, arrays of the present invention can be assembled to provide high-throughput processing, as follows.

FIG. 7 illustrates a plurality of systems 10 operating in parallel. Specifically, systems 10A, 10B and 10C are shown. An enlarged view of system 10A is provided. System 10A includes an input reservoir 20, a channel 25, a first output reservoir 30A and a second output reservoir 30B. The overall system operates as described above, however, a cell (or other particle) sorter system is also provided as follows. The sorting system illustrated herebelow sorts cells between first and second output reservoirs. It is to be understood that additional output reservoirs could be added. Thus, particle sorting among, three, four or more output reservoirs can be accomplished using the techniques as described herein.

When a desired particle has been identified in channel 25, a pressure differential is created across reservoirs 20 and 30A, thereby moving the desired particle into output reservoir 30A. Conversely, when a non-desired particle has been identified in channel 25, a pressure differential is created across reservoirs 20 and 30B, thereby moving the non-desired particle into output reservoir 30B. Thus, one-by-one sorting of the individual particles can be accomplished. As can be seen, the present invention is therefore able to both identify particles of interest, and also to sort such particles between different reservoirs, as desired. This is very advantageous since a large population of particles (having a low concentration of desirable particles) can be sequentially passed through system 10 with the present invention sorting such particles one-by-one and positioning them at a preferred location (e.g.: output reservoir 30A) for further analysis. The electrode leads of an exemplary micro-Coulter counter system (as set forth in the Sohn system, supra) are illustrated collectively as element 40.

A computer system 50 may be provided to control the operation of each of systems 10A, 10B and 10C. Computer system 50 may be used together with electrical detection system 40 to simultaneously measure the periods of time during which individual particles moves through each of the channels 25 in each of systems 10A, 10B and 10C.

In optional embodiments of the present invention, each of 10A, 10B and 10C may either have functionalized channels 25 as seen in FIG. 3 (to identify target particles) or may have non-functionalized channels 25 as seen in FIG. 5 (to determine the size of target particles). Any number of combinations of such systems may be provided, all keeping within the scope of the present invention.

Turning next to FIG. 8, a plurality of systems 10A, 10B, 10C, 10D, etc. may be operated in series. In such an embodiment of the present invention, particle identification, sizing and sorting can be accomplished as described above. In accordance with this particular embodiment of the invention, however, the output of one system 10 can be used as the input to another system 10. For example, cells C1, C2, C3 and C4 may initially be placed into input reservoir 20. System 10A then sorts cells C1 and C2 into reservoir 30A, and cells C3 and C4 into reservoir 30B, using a process as described above. System 10B then sorts cells C1 into reservoir 30C and cells C2 into reservoir 30D. System 10C similarly sorts cells C3 into reservoir 30E and cells C4 into reservoir 30F For ease of illustration, the output reservoir of one system is shown as being the same as the input reservoir of a second system. It is to be understood that although the output reservoir of one channel may be directed into the input reservoir of a second system, such two reservoirs may be one and the same; or they may instead be separated by a channel.

In one exemplary embodiment of the invention, system 10A is a sizing system (i.e. its channel 25 is not functionalized) and systems 10B and 10C are identification systems (i.e. their channels 25 are functionalized). As such, the system for determining the size of an individual particle is disposed upstream of the system for identifying individual particles. It is to be understood that such an example is merely exemplary and that an infinite number of combinations of microfluidic systems are possible by combining the parallel processing system illustrated in FIG. 7 with the series processing system illustrated in FIG. 8.

Applications of the Present Invention:

The applications of the present invention are practically limitless. Thus, the following represents a selection of exemplary uses which should not be taken as limiting of the invention in any way.

Potential applications for the present invention include characterization of cancer and other types of cells. This may include characterizing the cell surface expression of numerous proteins in acute leukemia cells (e.g., in acute myeloid leukemia, CD33, CD34, CD117; and in acute lymphoblastic leukemia, TdT, sIg). When functionalized channels 25 arranged in series (FIG. 8) the present invention can be used to perform immunophenotyping for acute myeloid and lymphoid leukemias.

The present invention also has the ability to perform immunophenotyping at the bedside. This would allow acute leukemic patients to be diagnosed immediately upon presentation, whether at a community health center, local doctor's office, or over the weekend or at night, when hematopathologists may not be available. Currently, there are no commercial point-of-care technologies available to perform complete blood count analysis or to diagnose or monitor cancer. Thus, the present invention could be used to advance point-of-care service. In addition, the present invention could be used to measure patient responses to cytotoxic chemotherapy by measuring the degree of chemotherapy-induced apoptosis.

Other applications include the isolation of circulating tumor and/or endothelial cells from patients with solid tumors and/or the isolation of circulating hematopoietic precursor cells from patients. For example, acute promyelocytic leukemia is typically CD33+, CD13+, HLA-DR−, CD117−, CD15−, CD11b−, and CD34−; and B-cell acute lymphoblastic leukemia (ALL) cells are TdT+, HLA-DR+, and often CD24+, surface immunoglobulin (SIg)−, and CD20−. Early precursor B-cell ALL cells are also CD19+, and common ALL cells are CD10+.

Experimental Results:

The Applicants have built and successfully operated the present invention. Details of such fabrication, and experimental results achieved are set forth below. It is to be understood that the following description sets forth exemplary processes and systems, and does not limit the scope of the present invention in any way.

(i) Device Fabrication

System 10 (as seen in FIG. 1) was built on a glass slide with patterned electrodes thermally bonded to a polydimethylsiloxane (PDMS) slab embedded with channel 25 and reservoirs 20 and 30. A 1.8 μm thick layer of AZ 3318D resist (Clariant) was first patterned using traditional photolithography. Thin-film deposition techniques were used to evaporate the Ti/Pt electrodes (5/25 nm). To generate channel 25 and reservoirs 20 and 30, a negative relief master was created on a Si wafer using UV lithography and two layers of SU8 negative resist (Microchem). A Ti/Au layer (5/10 nm) was first evaporated on a cleaned Si wafer to help aligning the pore with reservoirs 20 and 30. A layer of SU8 2015 resist was then span and patterned to generate the negative of channel 25 and the marks used to align the second layer of SU8. Measurements were performed with two shapes of channels: a 400 μm long×20 μm wide×20 μm high straight pore (FIG. 2A) and a 20 μm wide×20 μm high pore serpentine pore (FIG. 2B). After etching of the gold, a second layer of SU8 2015 resist was used to fabricate the negative of two 2000 μm long×600 μm wide and 30 μm high reservoirs 20 and 30. Polydimethylsiloxane (PDMS) (10:1 prepolymer:curing agent) was then dispensed onto the master and cured for at least 12 hours at 80° C. After cutting and removing the PDMS slab embedded with channel 25 and reservoirs 20 and 30, inlet and outlet holes 22 and 32 were punched using a 16 G syringe needle. The PDMS slab was then sealed with the patterned glass slide that has been already treated chemically as described in Device Functionalization, below.

(ii) Device Functionalization

To functionalize a glass substrate, the substrate was first treated with an oxygen plasma. Micro-contact printing was used to wet the substrate with a solution of aminopropyl triethoxysilane (APTES) in anhydrous toluene (10% weight) at room temperature, thereby coating the substrate surface with amino-silane groups. To ensure a stable APTES layer, the substrate was baked in an oven at 80° C. for 4-5 hours, thereby cross-linking the APTES. The cured substrate was soaked first in toluene (10 min) and then in deionized water (10 min for two times) to remove any unbound APTES. Once the APTES was patterned onto the glass substrate, a micropipette was used to apply a droplet (2-4 μL) of a 1 mM solution of N-5-Azido-nitrobenzoyloxysuccinimide (ANB-NOS) in HEPES (pH 7.3) to the area between the electrodes. After an overnight incubation, excess ANB-NOS was removed by washing the substrates with HEPES (10 min) and then rinsing with deionized water. A hot plate was used (10 min at 65° C. and 15 min at 150° C.) to seal the PDMS mold of the device onto the substrate. Antibodies were then injected into the pore and allowed to incubate for 3 hours. For the covalent binding of antibodies with the ANB-NOS treated glass substrate, a UV-light source (Ushio 350DS, 3 min) was used that activated the aryl azide photoreactive groups.

(iii) Fluid Handling And Data Acquisition

Pressure-driven flow inside wet micro-fluidic channels generated by a commercial micro-fluidic pump (Fluidigm, South San Francisco, Calif.). The device was connected with the pump using Tygon Microbore tubing (0.06″ o.d. and 0.02″ i.d.).

Electronic measurements were performed using a four-terminal apparatus in order to separate the electrical resistance of the electrolyte solution between the electrodes from the resistance of the electrodes themselves. During data acquisition, a constant voltage was applied between the inner electrodes (typically 0.7 V) using a Stanford Research Systems DS345 function generator, while the current is sampled at 50 kHz with a National Instruments PCI-6035E card after amplification (DL Instruments 1211) [11].

(iv) Cell Types And Culture Conditions:

Murine erythroleukemia (MEL) cells were grown in RPMI-1640 (Invitrogen) and 10% (v/v) fetal bovine serum (FBS) (Hyclone) at 37° C. and 5% CO₂. Cells were maintained at an average cell density of 2×10⁵ cells mL⁻¹. The cells used for apoptosis detection were IL-3 dependent primitive myeloid murine 32D cells and primary BALB/c mouse thymocytes from animals less than 9 weeks old. 5×10⁵ 32D cells were grown at 37° C. in Petri dishes to avoid cell adhesion. The growth medium consisted of RPMI with 10% (v/v) fetal bovine serum (FBS) and 10 ng ml⁻¹ of IL-3 (R&D Systems).

(v) Apoptosis Induction:

Apoptosis was induced in the 32D cells by IL-3 deprivation from the culture medium. Apoptosis detection was performed with the pore device after 24, 48, 72 and 96 hrs. Primary mouse thymocytes were incubated at 37° C. typically for 4-5 hours in a medium (RPMI with 10% (v/v) FBS) containing 0.5 μg mL⁻¹ anti-CD95 monoclonal antibody (clone RK-8) (Abcam) to induce apoptosis.

(vi) Flow Cytometer Measurements

The presence of CD34 receptors on murine erythroleukemia (MEL) cells and the affinity of the CD34 antibody (eBioscience) for the matching receptor were first tested. 5×10⁵ murine erythroleukemia (MEL) cells were first washed in 1 mL of PBS and then were resuspended in 1.2 ml CD34-FITC antibody solution (0.033 μg ml⁻¹). Incubated cells were then measured with conventional flow cytometry within 30 min and compared to murine erythroleukemia (MEL) cells incubated without anti-CD34 antibody.

Apoptosis was detected with Annexin V-FITC conjugates (R&D Systems) which bind phosphatidylserine (PS) translocated to the outer leaflet of the membrane of apoptotic cells. Approximately 2×10⁵ 32D cells or primary thymocytes were first washed in 1×PBS and then resuspended in 100 μl of fresh buffer containing Annexin V (0.25 μg ml⁻¹). After 10 min incubation, 400 μl of buffer (10 mM Hepes, 0.15 M NaCl, 5 mM KCl, 1 mM MgCl₂, 1.8 mM CaCl₂) was added. All incubation steps were performed in the dark. Apoptosis was measured by flow cytometry (Beckman Coulter) within half an hour after the incubation process.

(vii) Results And Discussion

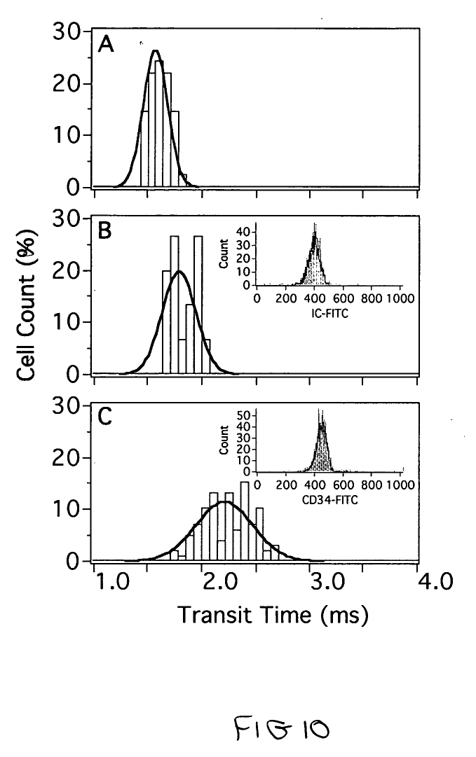

Based on the relationship between pulse magnitude and cell diameter, a device with an unfunctionalized straight pore (FIG. 9A) with rectangular cross-section was used to determine cell size very accurately using the formulation proposed by Maxwell for a cylindrical pore and assuming that

$$\vert \frac{δ R}{R} \vert \approx \vert \frac{δ I}{I} \vert ,$$

(Eq. 1). Eq (1) was validated by first flowing colloids of known precise diameters (4.9-15.03 μm diameter) 4.9 μm (Interfacial Dynamics), 9.86 μm (Bangs Laboratories), and 15.03 μm (Duke Scientific) through the device:

$$\frac{δ I}{I} = \frac{{d}^{3}}{{D}^{2} L} \qquad Eq . ( 1 )$$

where d is the diameter of the cell/colloid, and D and L are the diameter and the length of the pore respectively. FIG. 9A shows a comparison between the measured mean pulse magnitude and those predicted by Eq. (1) The error of the measured colloid diameter is less than 10% of the nominal colloid size, which makes Eq. (1) a good model for cell size analysis with the present invention.

The ability of the present invention to distinguish two cell types within a mixed cell population at varying concentrations was then tested. FIG. 9B shows a representative current vs time trace obtained when a mixture of primary mouse thymocytes (6 μm diameter) and mouse erythroleukemia (MEL) cells (8-15 μm diameter) were injected into an unfunctionalized, straight pore at 21 kPa (3 psi). Each downward pulse corresponds to a single cell passing through the pore. A stable square pulse shape was measured easily with the time scale used to obtain these measurements (˜50 μs) (FIG. 9B inset). FIG. 9C shows the resulting cell-size distribution when we apply Eq. (1) to the measured pulse magnitudes given in FIG. 9B. The cell population designated as “1” in the figure corresponds to the thymocytes, and the cell population designated as “2” corresponds to the MEL cells. FIGS. 9D and 9E show the cell size distributions obtained when MEL cells are mixed with primary mouse thymocytes at different percentages, and the insets show excellent agreement with similar measurements made using forward scattering in traditional flow cytometry (Beckman Coulter) (FIGS. 9C-9E inset). FIG. 9D shows the cell size distribution when 57.2% of the cells are primary mouse thymocytes and 42.8% of the cells are MEL cells. The present invention was able to detect 2 MEL cells out of a population of 200 cells examined (FIG. 9E), thus demonstrating the exquisite sensitivity of the present invention. Because of this high sensitivity, the present invention generated reliable data using fewer than 500 cells, approximately an order of magnitude less than that required by flow cytometry. This demonstrated that the present invention is applicable to the detection of rare events.

The present invention was also able to distinguish cell types in a population on the basis of cell size, to quantify the number of apoptotic cells in a population. Murine myeloid 32D cells are IL-3 dependent and undergo apoptosis when starved of IL-3. The present invention and traditional flow cytometry were used to measure the percentage of apoptotic versus viable cells by depriving 32D cells of IL-3 for increasing amounts of time (FIGS. 9F-9H). Apoptotic cells (designated as “1” in the figure) shrink and flatten, resulting in measured pulses that are smaller in magnitude as compared to those obtained with viable cells (designated as “2” in the figure). After 24 hrs of IL-3 deprivation, 4.2% of the 32D cells were apoptotic (FIG. 2F), after 72 hrs, 21.5% (FIG. 2G), and after 96 hrs, 44.2% (FIG. 9H). These percentages agree well with those obtained for the same sample of cells using traditional flow cytometry (inset).

As can be seen, the present invention has the ability to measure cell size very accurately. By combining the resistive-pulse sensing technique with a pore that was functionalized with antibodies having high specificity for a cell-surface marker of interest, the present invention was able to characterize markers present at the cell surface without the need for addition of exogenous labels. Specifically, channel 25 was functionalized with antibodies having high specificity for the cell surface marker of interest. Many techniques to accomplish this are covered by the present invention. In one specific example, the chemistry used to attach the antibodies to the inner walls of channel 25 consists of three steps: First, microcontact printing was used to coat amino-silane groups in the region between the electrodes on the glass substrate. Second, a hetero-bifunctional cross-linker (ANB-NOS) was coupled to the amino-silane groups through incubation. Third, antibodies were attached covalently to the ANB-NOS cross-linker through the activation of the aril-azide group using UV light. The link between the antibodies and the cross-linker was achieved by incubation of antibodies inside the pore after the device fabrication. Incubation also allowed the adsorption of antibodies on the PDMS walls of the pore. An antibody concentration of 2-3 μg/ml was used typically in this last step, and during incubation, the PDMS pore walls absorb the antibodies. Concentrations above 10 μg/ml result in more prolonged cell/antibody interactions and an increased the likelihood of clogging the pore.

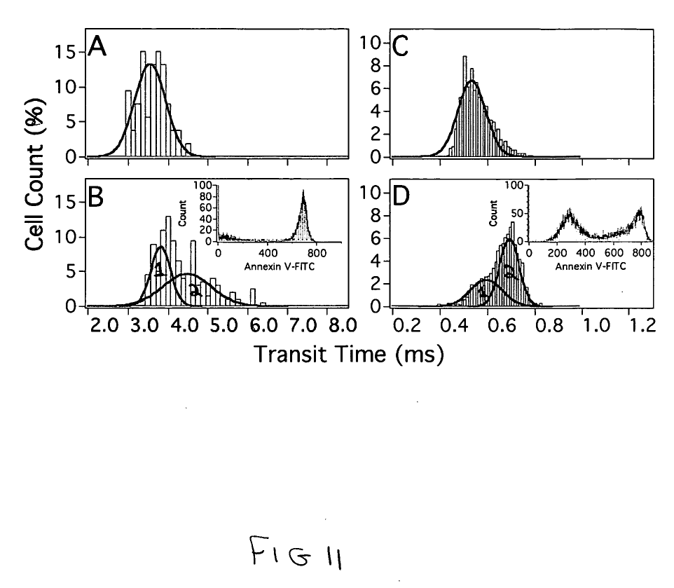

To screen particular cell-surface markers on individual cells, a serpentine-shaped pore (FIG. 2B) was used, because the geometry offered an increased surface area with which individual cells could interact with the functionalized antibodies (FIG. 3). CD34 receptors expressed on the surface of MEL cells were detected, and a consistent pressure was used to drive cells across the channel. The cell transit time (i.e. pulse width) distributions were measured for three different pores: a non-functionalized or “blank channel” (i.e. one that had not been functionalized with any antibody) (FIG. 10A); a channel functionalized with an isotype-control antibody (FIG. 10B); and a channel functionalized with an anti-CD34 antibody (FIG. 10C). As shown in FIG. 10, the average time the cells take to pass through the different pores: 1.57±0.16 ms through the blank channel; 1.79±0.22 ms through the isotype-control antibody channel; and 2.22±0.37 ms through the anti-CD34 antibody channel, indicating a clear increase when the specific antibody was used. The slight increase in average time for MEL cells to pass through a channel functionalized with an irrelevant antibody as compared to a blank channel is due to the nonspecific interactions between the cell surface and the antibodies on the channel walls. The significant increase in average time for MEL cells to flow through a channel in which anti-CD34 antibodies are present is due to the high affinity between the antibodies functionalized on the channel walls and the receptors on the cell surface (FIG. 10C).

As a second demonstration of the present invention's ability to screen cells based on cell-specific markers, a technique to detect apoptotic cells was used. In contrast to the previous experiment in which cell size was used to screen for apoptotic cells, annexin V's ability to bind to the negatively-charged phosphatidylserine residues that become localized to the outer leaflet of the cell membrane during apoptosis to screen cells was used. This “indirect” apoptosis assay involves incubating cells with annexin V and then injecting them through a serpentine channel functionalized with anti-annexin V antibody. For controls, cells were injected through a blank pore and a FACS analysis was performed with the same solution of cells.

Compared to the previously-described MEL cells experiments, here we have two populations of cells: cells that are apoptotic and bind annexin V, and cells that are viable and do not bind annexin V. The present invention can differentiate between these two cell populations with a channel functionalized with anti-annexin V antibody: cells that are apoptotic and bind annexin V travel through the pore more slowly than viable cells which do not bind annexin V. FIG. 11A shows the normalized time distribution derived from a blank channel of a mixture of viable and apoptotic primary mouse thymocytes. As shown, the blank channel cannot discriminate between the viable and apoptotic cells. In contrast, FIG. 11B shows two distribution of cells, corresponding to the viable cells (designated as “1” in the figure) and apoptotic ones (designated as “2” in the figure). As shown, 42% of the cells are viable and 58% are apoptotic, agreeing well with the distributions derived from FACS analysis (inset). FIGS. 11C and D are normalized time distributions of murine 32D cells deprived from IL-3 from the culture medium to induce apoptosis. Again, the blank channel cannot discriminate between viable and apoptotic cells (FIG. 11C) while the functionalized pore can. 40% of the murine 32D cells are viable (labeled as “1” in the figure), whereas 60% are apoptotic (labeled as “2” in the figure). A similar distribution was obtained with flow cytometry.

The present invention is a powerful tool with which one can screen individual cells based on size or the expression of cell-surface markers. One advantage of the present invention is that it is a label-free technology that could be applied to any cell surface marker which binds another molecule that can be functionalized onto the channel walls. As can be seen, the present invention can be used to screen the CD34 marker on leukemia cells.

In one exemplary use of the present invention, leukemia cells can be immunophenotyped by injecting unlabeled cells into a device consisting of a series of channels, each functionalized with different and specific antibodies (e.g. CD34 , CD33, CD13, HLA-DR). The microfluidics platform upon which the present invention can operate offers flexibility in terms of the possibility of isolating cells after interrogation. Because the unlabeled cells are not damaged during interrogation, they may be cultured in bioreactors on the same chip and subjected to drug screening or biomarker discovery. Finally, phenotyping disease simply, rapidly, and on a single microchip platform would represent a paradigm shift in point-of-care diagnostics.

Moreover, although there is an excellent match between the distributions obtained with our pore device and with a flow cytometer, the advantage of the present invention is that it does not require calibration if different cells are measured because the magnitude of the resistive pulses depend only on the cell characteristics and on the geometry of the channel. Furthermore, this device presents the opportunity for point-of-care diagnostics.

What is claimed:

1. An apparatus for identifying individual particles, comprising:
an input reservoir;
at least one output reservoir;
a channel connecting the input reservoir to the at least one output reservoir, wherein the channel is functionalized with at least one molecule selected to interact with a marker on a surface of a particle;
a system to move fluid containing the particle from the input reservoir through the channel and into the at least one output reservoir; and
a system to measure the period of time during which the particle moves through the channel.

2. The apparatus of claim 1, wherein the particle is a cell.

3. The apparatus of claim 1, wherein the particle is a cell fragment.

4. The apparatus of claim 1, wherein the particle is a colloid.

5. The apparatus of claim 1, wherein the particle is selected from the group consisting of a bacterium, a virus, a fungus, a micelle, a liposome, DNA or RNA or an oligonucleotide chain.

6. The apparatus of claim 1, wherein the at least one molecule with which the channel is functionalized is a protein.

7. The apparatus of claim 1, wherein the at least one molecule with which the channel is functionalized is selected from the group consisting of a phospholipid, a sugar, a carbohydrate, a peptidoglycan, DNA, RNA or an oligonucleotide chain.

8. The apparatus of claim 1, wherein the marker on the surface of the particle is a protein.

9. The apparatus of claim 1, wherein the marker on the surface of the particle is selected from the group consisting of a phospholipid, a sugar, a carbohydrate, a peptidoglycan, DNA or RNA or any oligonucleotide chain.

10. The apparatus of claim 1, wherein the channel is a straight channel.

11. The apparatus of claim 1, wherein the channel is a serpentine channel.

12. The apparatus of claim 1, wherein the system to measure the period of time during which the particle moves through the channel comprises a system for measuring a change in electrical resistance across the channel.

13. The apparatus of claim 1, wherein the system to measure the period of time during which the particle moves through the channel comprises a system for measuring current change across the channel over time.

14. The apparatus of claim 13, wherein the system for measuring current change across the channel over time comprises a Coulter counter.

15. The apparatus of claim 11, wherein the fluid is a conducting fluid.

16. The apparatus of claim 1, wherein the channel has a width of less than 50 μm.

17. The apparatus of claim 1, wherein the channel has a length of less than 2 cm.

18. The apparatus of claim 1, wherein the input reservoir, the channel and the at least one output reservoir are all fabricated into a unitary block of material.

19. The apparatus of claim 18, wherein the unitary block of material is selected from the group consisting of PDMS, glass, quartz, a plastic substrate, silicon, and a semi-conductor wafer.

20. The apparatus of claim 1, wherein the at least one output reservoir comprises first and second output reservoirs, further comprising:
a particle sorter configured to direct the particle to either the first output reservoir or the second output reservoir based on identification of the particle.

21. A system for parallel identification of individual particles, comprising:
(a) a first apparatus for identifying individual particles, comprising:
an input reservoir;
at least one output reservoir;
a channel connecting the input reservoir to the at least one output reservoir, wherein the channel is functionalized with at least one molecule selected to interact with a marker on a surface of a particle;
a system to move fluid containing the particle from the input reservoir through the channel and into the at least one output reservoir;
(b) a second apparatus for identifying individual particles, comprising:
an input reservoir;
at least one output reservoir;
a channel connecting the input reservoir to the at least one output reservoir, wherein the channel is functionalized with at least one molecule selected to interact with a marker on a surface of a particle; and
a system to move fluid containing the particle from the input reservoir through the channel and into the at least one output reservoir; and
(c) a system to simultaneously measure the periods of time during which the particle moves through the channel in each of the first and apparatus and the second apparatus.

22. The system of claim 22, further comprising:
a computer configured to simultaneously control the operation of the first apparatus and the second apparatus.

23. The system of claim 22, wherein the at least one output reservoir in each of the first and second apparati comprise first and second output reservoirs, and wherein each of the first and second apparati further comprise:
a particle sorter configured to direct the particle to either the first output reservoir or the second output reservoir based on identification of the particle.

24. An apparatus for determining the size of an individual particle, comprising:
an input reservoir;
at least one output reservoir;
a channel connecting the input reservoir to the at least one output reservoir, wherein the channel is filled with a conducting fluid;
a system to move fluid containing a particle from the input reservoir through the channel and into the at least one output reservoir;
a system for measuring a change in electrical resistance across the channel; and
a system for correlating the amplitude of the change in electrical resistance across the channel to the size of the particle.

25. The apparatus of claim 24, wherein the system to measuring a change in electrical resistance across the channel comprises a system for measuring current change across the channel.

26. The apparatus of claim 24, wherein the system to measuring a change in electrical resistance across the channel comprises a Coulter counter.

27. The apparatus of claim 24, wherein the particle is a cell or cell fragment.

28. The apparatus of claim 24, wherein the particle is a colloid.

29. The apparatus of claim 24, wherein the particle is selected from the group consisting of a bacterium, a virus, a fungus, a micelle, a liposome, DNA, RNA or any oligonucleotide chain.

30. The apparatus of claim 24, wherein the channel has a width of less than 50 μm.

31. The apparatus of claim 24, wherein the channel has a length of less than 2 cm.

32. The apparatus of claim 24, wherein the at least one output reservoir comprises first and second output reservoirs, further comprising:
a particle sorter configured to direct the particle to either the first output reservoir or the second output reservoir based on the size of the particle.

33. A system for sizing and identifying individual particles, comprising:
(a) an apparatus for determining the size of an individual particle, comprising:
an input reservoir;
at least one output reservoir;
a channel connecting the input reservoir to the at least one output reservoir, wherein the channel is filled with a conducting fluid;
a system to move fluid containing a particle from the input reservoir through the channel and into the at least one output reservoir;
a system for measuring a change in electrical resistance across the channel; and
a system for correlating the amplitude of the change in electrical resistance across the channel to the size of the particle; and
(b) an apparatus for identifying individual particles, comprising:
an input reservoir;
at least one output reservoir;
a channel connecting the input reservoir to the at least one output reservoir, wherein the channel is functionalized with at least one molecule selected to interact with a marker on a surface of a particle;
a system to move fluid containing the particle from the input reservoir through the channel and into the at least one output reservoir; and
a system to measure the period of time during which the particle moves through the channel, wherein the apparatus for determining the size of an individual particle is in fluid communication with the apparatus for identifying individual particles.

34. The system of claim 33, wherein the apparatus for determining the size of an individual particle is positioned upstream of the apparatus for identifying individual particles.

35. A method of identifying individual particles, comprising:
passing a particle through a microfluidic channel functionalized with at least one molecule selected to interact with a marker on a surface of the particle; and
identifying the particle by determining the period of time during which the particle moves through the microfluidic channel.

36. The method of claim 35, wherein the molecule functionalized onto the microfluidic channel interacts with the marker on the surface of the particle so as to slow passage of the particle through the microfluidic channel.

37. The method of claim 35, wherein the method of determining the period of time during which the particle moves through the channel comprises:
measuring a change in electrical resistance across the microfluidic channel over a period of time.

38. The method of claim 35, further comprising:
sorting the particle from other objects passing through the microfluidic channel after the particle has been identified.

39. A method of sizing individual particles, comprising:
passing a particle through a microfluidic channel;
measuring a change in electrical resistance across the microfluidic channel; and
correlating the amplitude of the change in electrical resistance across the microfluidic channel to the size of the particle.

40. The method of claim 39, further comprising:
sorting the particle from other objects passing through the microfluidic channel after the particle has been sized.
